(19) United States
(12) Patent Application Publication
(10) Pub. No.: US 2026/0102601 A1
(43) Pub. Date: Apr. 16, 2026

(54) INTRALUMINAL PRESSURE DAMPENING AND ENERGY RECOIL DEVICE

(71) Applicant: QHEART MEDICAL PTY LTD, Taringa (AU)

(72) Inventor: Peter WALSH, Brisbane (AU)

(21) Appl. No.: 19/117,221

(22) PCT Filed: Oct. 2, 2023
(86) PCT No.: PCT/AU2023/050946

§ 371 Date: Mar. 31, 2025

(30) Foreign Application Priority Data
Oct. 1, 2022 (AU) 2022902845
Sep. 30, 2023 (AU) 2023903149

(51) Int. Cl. A61M 60/139; A61M 25/10; A61M 60/295; A61M 60/865

(52) U.S. Cl. CPC A61M 60/139; A61M 60/295; A61M 60/865; A61M 2025/1004; A61M 2025/1013; A61M 25/10184; A61M 2205/04; A61M 2205/3331

(57) ABSTRACT

The present invention is a device for improving the aortic vessel recoil and overcoming aortic stiffness to provide a therapy to improve heart function and decrease high blood pressure, said device being an Intraluminal Pressure Dampening and Energy Recoil Device (IPDERD), an implantable device within a tubular or sac wall of said vessel in a human or animal body, said device including a changeable volume portion which is adapted to interact with said vessel so as to modify the volume within said vessel; and an energy storage means functioning with said changeable volume portion whereby a decrease in the volume of said changeable volume portion creates a pressure or energy charge in said energy storage means, said pressure or energy charge being able to be subsequently released to cause said changeable volume portion to increase in volume, said device including a fixation feature such as a stent for fixation and a changeable volume feature such as a balloon for dampening and counterpulsating. The present invention further provides a method of treating a vessel in a human or animal body, said method including the steps of: preparing a patient; identifying a site in said vessel requiring treatment; positioning an Intraluminal Pressure Dampening and Energy Recoil Device (IPDERD), an implantable device within a portion of tubular or sac wall of said vessel at said site.

### CROSS-REFERENCE TO RELATED APPLICATIONS

This application is a national phase application of International Application No. PCT/AU2023/050946 entitled “NTRALUMINAL PRESSURE DAMPENING AND ENERGY RECOIL DEVICE,” filed Oct. 2, 2023, which claims the benefit of and priority to Australian Patent Application No. 2022902845 filed Oct. 1, 2022, and Australia Patent Application No. 2023903149 filed Sep. 30, 2023, the contents of all of which being incorporated by reference in their entireties herein.

### TECHNICAL FIELD

The present disclosure relates to tubular wall compliance and load bearing devices and methods for their deployment within human and or animal bodies, so as to change or modify the compliance or the load bearing capacity in responses to pulsatile pressure loading of a tubular or sac wall section. The present disclosure consists of an intraluminal stent or fixation component and one or more balloons, reservoirs, or chambers, connected with a catheter or tubing line for adjustment and monitoring, which are deployed in a crimped state to the vessel treatment site where they are unloaded to attach and act intraluminally within the lumen of the vessel.

When applied to the cardiovascular system, these disclosures serve to boost the secondary heart pump action of the heart, by dampening the time dependent blood pressure profile during systole, and enhancing the time dependent blood pressure profile during diastole, thereby reducing heart load and improving aortic and coronary artery blood flow.

The inclusion of additional balloons, reservoirs or chambers allows the device to treat more vessels along the aorta from the ascending, thoracic, and abdominal aorta and to provide a pulse pressure loading reduction by dampening and reducing systolic blood pressure and boosting diastolic pressure thereby improving coronary, carotid, and renal artery blood flows.

### BACKGROUND

Heart failure is the fastest growing cardiovascular disorder. Incidence is rising at a rate of approximately 2% to 5% in people over 65 years of age, and 10% in people over 75 years of age.

Heart failure is a leading cause of hospital admissions and re-admissions in Americans older than 65 years of age.

Hypertension is a common condition prior to heart failure. In a recent study; 91% of people who developed heart failure had previous hypertension, of which 42% had systolic dysfunction and 58% had diastolic dysfunction.

Aortic stiffening, due to elastin degradation and other forms of stiffening, such as that caused by atherosclerosis, which is stiffening due to the presence and build-up of plaques, are a cause of hypertension. The aorta stiffens and dilates with age increasing: the load on the heart; pressure in left ventricle; aortic pressure at the time of peak aortic flow, and pulse wave velocity in the aorta and early wave reflection thus increasing pressure in late systole.

Data shows that systolic blood pressure continues to rise with age and diastolic pressure remains constant after approximately 50 years of age, giving an increase in pulse pressure after 50 years of age.

As the aorta stiffens, the arterial system suffers from a lack of compliance, leading to hypertension. Therefore, aortic stiffening appears to be a factor leading to high blood pressure (hypertension), heart failure, stroke, and renal disease.

Aortic compliance is fundamental to effective cardiovascular dynamics. Lack of aortic compliance leads to increased heart loading during systole and poor coronary artery perfusion during diastole due to a lack of vessel recoil. Decreases in aortic compliance occur with age as a result of stiffening in the aortic wall.

Approximately 80% of arterial compliance is in the ascending aorta and aortic arch sections. This expansion during systole and contraction/recoil during diastole of the ascending aorta and arch, is referred to as the secondary heart pump; an action that decays with age a directly associated with reduced compliance and increase aortic stiffness.

Stiffness of the aortic wall can be defined using various measures, and is commonly expressed as the pressure-strain elastic modulus, E_{P}:

$${E}_{P} = {D}_{dia} \times ( {D}_{s y s} - {D}_{d i a} ) / ( {P}_{s y s} - {P}_{d i a} ) .$$

Where D_{sys} and D_{dia} and the diameter of the vessel in systole and diastole respectively, and P_{sys} and P_{dia} are the pressure within the vessel at systole and diastole respectively.

Pulse Pressure (PP) is the difference between P_{sys} and P_{dia}, the pressure within the vessel at systole and diastole respectively.

Systolic and diastolic blood pressures continue to increase with age, up to around 50 years of age, thereafter systolic blood pressure continues to increase while diastolic blood pressure does not, this increased pulse pressure increasing heart load, mean blood pressure, and decreases blood flow during diastole to adjoining vessels including the coronary arteries to the heart.

Previous solutions for addressing heart failure include:
(a) medications which have limited benefits and generally high costs associated with them:
(b) intra-aortic balloon pumps (IABPs) which deflate during systole to reduce pressure and counterpulsate during diastole to augment pressure, however this is only a temporary solution due to patients being bed bound and connected to a large external console;
(c) ventricular assist devices, extraluminal and intraluminal compression devices and pumps, which require power sources thereby increasing complexity of implanting, increased expense and have higher risk to the patient, and
(d) heart transplants which are limited by availability, high cost and high risk.

To treat vessel stiffness, adding a compliant chamber secured within or outside of a vessel can provide Aortic Recoil Repair therapy leading to reduced blood pressure, reduced heart load, increased stroke volume, and increased cardiac output. Aortic Recoil Repair in devices made by the applicant have been tested in pig and human studies showing an increase in cardiac output by 30% in pigs and 25%-29% in humans.

The applicant does not concede that the prior art discussed in the specification forms part of the common general knowledge in the art at the priority date of this application.

### BRIEF SUMMARY

The present disclosure is a device for improving the aortic vessel recoil and overcoming aortic stiffness to provide a therapy to improve heart function and decrease high blood pressure, said device being an Intraluminal Pressure Dampening and Energy Recoil Device (IPDERD), an implantable device within a tubular or sac wall of said vessel in a human or animal body, said device including a changeable volume portion which is adapted to interact with said vessel so as to modify the volume within said vessel; and an energy storage means functioning with said changeable volume portion whereby a decrease in the volume of said changeable volume portion creates a pressure or energy charge in said energy storage means, said pressure or energy charge being able to be subsequently released to cause said changeable volume portion to increase in volume, said device including a fixation feature such as a stent for fixation and a changeable volume feature such as a balloon for dampening and counterpulsating.

Said device can have intraluminal components, said intraluminal components being compressed and crimped into a deployment sheath for deployment into a treatment vessel using an interventional access procedure via an adjoining vessel to slide the device from the access vessel to the target vessel treatment site, where the device is unloaded from a deployment catheter sheath to expand to the diameter of the vessel to engage and fix the device at the target vessel location, said device having an inflation line being connected to an intraluminal adjustment port or a trans-vascular line to an enclosure allowing monitoring and adjustment and containing additional balloon, reservoir, or chambers to increase to devices dampening volume.

The changeable volume portion can be a balloon.

The energy storage means can be a pressure storage means such as a windkessel or deformable reservoir, or balloon.

The changeable volume portion can be constructed at least in part from an elastomeric material, said elastomeric material being said energy storage means.

The deformable reservoir, balloon, or stent, can have multiple elements in series or parallel to improve the overall performance.

The changeable volume portion and said energy storage means can be primed with a threshold or reference pressure and or volume. The volume and energy of the device can be modified after implant by using a subcutaneous port under the skin, which is attached to the changeable volume portion and energy storage device via a connected tube. A subcutaneous needle is inserted through the skin into the implanted subcutaneous port to add or remove volume.

The media with which the changeable volume portion can be primed with one or more of the following media: a bio-compatible fluid; liquid silicone; liquid saline; a liquid containing a contrast agent which is x-ray viewable; a gel or other solution that expands with temperature to a final operating volume at 37° degrees Celsius; elastin; collagen; elastin and collagen in combination; air; carbon dioxide; helium, or a gas, water, or an incompressible media.

The energy storage means can include a compressible fluid chamber.

Media with which said energy storage means can be primed is one or more of the following compressible media: nitrogen, air; carbon dioxide, helium, or gas, or other compressible media.

The device can be implanted using interventional techniques via femoral artery access, or subclavian access into the ascending, thoracic, and abdominal aortas, and can use an intraluminal adjustment port to change the operational pressure or charge by connecting to the intraluminal port via an intraluminal access line, or the device can have a trans-vascular line to an implanted module which has a subcutaneous adjustment port with a wireless monitoring system.

The device can be used to repair the compliance of a portion of said vessel.

The device can be used to modify the systolic and diastolic characteristics of said vessel to thereby improve cardiovascular performance.

The changeable volume portion can include electronic dynamic dampening control and energy harvesting and discharging means.

The device can be applied in the aorta.

The device can be applied in both the ascending aorta and the pulmonary artery.

The device can be applied to multiple vessels including the ascending and descending vessels attached to both the right and left sides of the heart.

The pressure or energy charge can be an energy charge which is at least in part produced by gas compression with an intraluminal balloon of said device.

The pressure or energy charge can be an energy charge which is at least in part produced by elastic deformation of said device.

Operation of said device can result in a system containing said vessel operating in a less stiff and or more compliant manner than would have been present from said portion of said wall at said site as untreated.

The energy storage device releases said pressure or energy charge to enable said device to assist said pressure within the vessel when said wall device acts upon said load.

The device can include at least one elastomeric component, said elastomeric component being adapted to release energy to assist said vessel.

The device or said energy storage means releases said pressure or energy charge in response to unloading of said vessel.

The present disclosure also provides a method of treating a vessel in a human or animal body, said method including: preparing a patient; identifying a site in said vessel requiring treatment; positioning an implantable device within a tubular or sac wall of said vessel at said site, whereby load applied to said vessel is borne by said wall and said device, said vessel being assisted by said device when said wall and said device acts upon said load, said device including an energy storage means which is charged with a pressure or energy charge by means of said load being applied to said device, said device including a stent for fixation and a balloon for dampening and counterpulsating, said device being implanted within the lumen of a vessel by loading of a compressed and crimped deployment catheter and removing a sheath to expand said stent within said vessel, said inflation line being connected to an intraluminal adjustment port or a trans-vascular line to an enclosure allowing monitoring and adjustment and containing additional balloon, reservoir, or chambers to increase the devices dampening volume.

The method can include positioning a stent, which is a part of said device, within said vessel.

The energy storage means can be a windkessel or deformable reservoir.

The energy storage means can include a compressible media chamber which when compressed stores said pressure or energy charge.

The energy storage means can include an electronic energy harvesting means.

The compliance of the device can be modified at the time of implant by inflation and or after implantation.

The compliance of the device can be modified after implant by using a subcutaneous port under the skin, which is attached to the changeable volume portion via trans-vascular access port and the energy storage means via a connected catheter tube. A subcutaneous needle is inserted through the skin to the implant port to add or remove volume.

The performance of the device can be monitored by an electronic sensor mounted in the subcutaneous port and or mounted in the changeable volume portion and energy storage means.

The sensor can be powered by an attached implanted battery, an attached implanted induction coil charged via inductive power delivered by an external coil, or via electrical power connected when an electrical subcutaneous port and power needle are used.

The sensor can be connected to an electronic communications circuit via analogue to digital conversion or via a digital connection. The electronic communication circuit can send data electronically via RF, wi-fi, or blue tooth to an external receiver to log and record data.

Compliance can be modified by inflation with one, or a combination of more than one, of the following media: a bio-compatible fluid; liquid silicone; liquid saline; a liquid containing a contrast agent (x-ray viewable); a gel solution that expands with temperature to a final operating volume at 37° degrees Celsius; uncured or liquid polymer which is thermosetting, at 37° C. or via activation by light or heat; a heat activated gel; elastin; collagen; elastin and collagen in combination; air; a polymer that cures or thermosets after injecting, gas, carbon dioxide, helium, or air or other compressible media, water.

The vessel can be a blood vessel.

The load applied to said vessel being borne by said wall and said device can be a systole phase of a cardiovascular system.

When said wall and said device acts upon said load it can be a diastole phase of a cardiovascular system.

### BRIEF DESCRIPTION OF THE DRAWINGS

An embodiment of the present disclosure will now be described, by way of example only, with reference to the accompanying drawings, in which:

FIG. 1 shows multi-balloon multi-stent Intraluminal Pressure Dampening and Energy Recoil Device (IPDERD) that contains an attached implantable enclosure with a subcutaneous port, an additional balloon or dampening chamber, and a wireless monitoring system, with the insert showing the wireless signal being received by a computer, phone, or watch device.

FIG. 2 shows an IPDERD with a trans-vessel catheter for monitoring and adjustment of the IPDER device, where the adjustment port on the attached module is being adjusted by the subcutaneous port with a syringe inserted into the port which can use a multi-lumen tubing connection.

FIG. 3 shows an IPDERD with an intraluminal port after the inflation and adjustment line has been removed.

FIG. 4 shows an IPDERD being adjusted by the attachment line being re-attached via a femoral artery access procedure using an intraluminal access line attached to the IPDERD's intraluminal port and adjusted with the attached syringe and external pressure meter.

FIG. 5 shows an IPDERD deployed via the left subclavian artery with the first balloon in the ascending aorta, a second balloon in the thoracic aorta, and a third balloon in the abdominal aorta.

FIG. 6 shows an IPDERD deployed via the femoral artery with the first balloon in the thoracic aorta and the second and third balloons in the abdominal and lower abdominal aorta.

FIG. 7 shows the collapsed and crimped IPDERD being deployed in the target vessel and released by the outer sheath being removed allowing the stent to engage the vessel wall followed by the release of the balloon and balloon inflation.

FIG. 8 shows a balloon and stent loaded into a deployment sheath.

### DETAILED DESCRIPTION

Multi-balloon with multi-stent Intraluminal Pressure Dampening and Energy Recoil Device (IPDERD) 1000

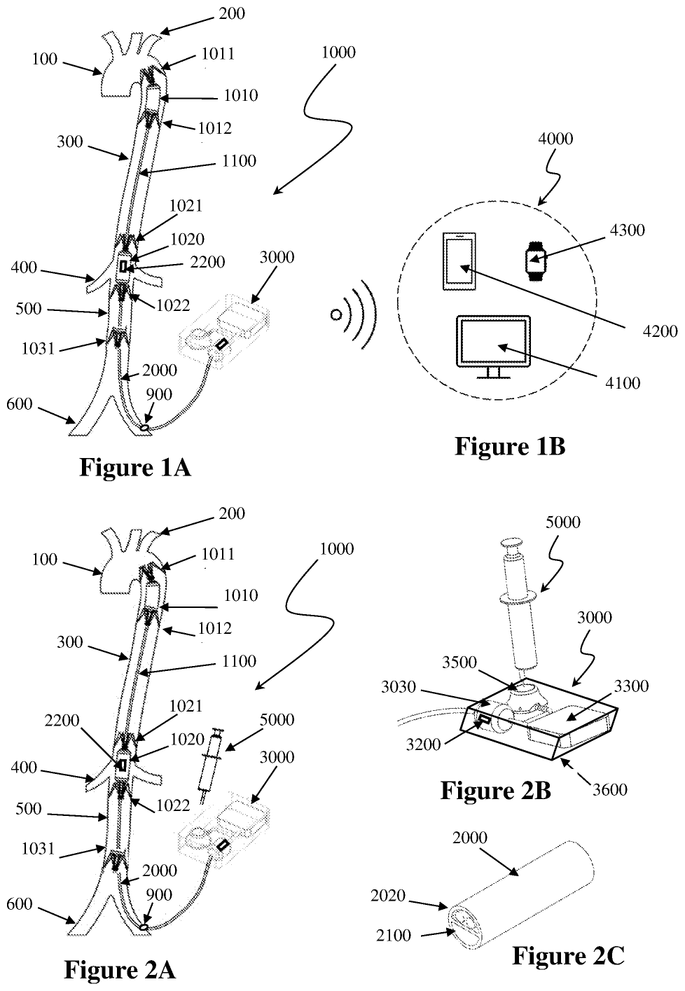

FIG. 1 shows a multi-balloon multi-stent Intraluminal Pressure Dampening and Energy Recoil Device (IPDERD) 1000.

The IPDERD 1000 consists of axial balloons 1010 and 1020, connected to a catheter tubing line 1100 and fixed within the aortic vessel 100, in the upper thoracic aorta 300 distal to the left subclavian artery 200 extending to the abdominal aorta 500 adjacent to the kidneys 400, with the balloons being held by stents 1011, 1012, 1021, 1022 and 1031.

### Trans-Vascular Line Attached to Implanted Enclosure for Monitoring and Adjustment of IPDERD 3000

The IPDERD 1000 is connected to an implantable enclosure 3000 via a catheter tubing line 2000, using trans-vascular access port 900, attaching sensor 2200 and the catheter tubing line 2000 to the implantable enclosure 3000, and as shown in the insert of FIG. 2 this consists of a subcutaneous inflation and adjustment port 3500, an additional balloon or dampening chamber 3030, and a wireless monitoring system 3300. Catheter tubing line 2000 (as shown in the second insert in FIG. 2) can have multi lumens allowing both media inflation 2100 and electronic connections 2020 to connect the enclosure 3000, and or other connection lines if so desired.

The FIG. 1 insert 4000 shows the wireless signal being received by a computer 4100, a phone 4200, or watch devices 4300.

The IPDERD is implanted using interventional techniques further described below with reference to FIG. 7.

Using port 3500 and subcutaneous needle and syringe 5000, the balloons are inflated to the mean pressure of the aorta, or other physician chosen pressure level. The wireless pressure monitor can log the pressure in the balloons. An external pressure monitor such as that shown in FIG. 4 5300 can also be used. The needle is removed and the IPDERD is now active and operational. The implant housing can be inserted in a skin pocket in the lower abdominal region and the patient can be recovered.

### Intraluminal Pressure Dampening and Energy Recoil Device (IPDERD) 1000

Once the balloons 1010, 1020, and 3200 are set to the operational pressure, the action of systolic pressure dampening and diastolic energy recoil occurs. As the pressure is systole increases and travels through the aorta, balloon 1011 reduces its volume by the compression of its media within and the higher pressure also allows this media to move downstream to the connected balloon 1020 and or chambers 3200, the result of which reduces the systolic blood pressure of the entire arterial left arterial system reducing left heart load. As the arterial pressure wave propagates and the diastolic phase occurs, the pressure reduces and balloon 1010 expands its volume by the media expanding and volume moving back from the attached balloons 1020 or chambers 3200, causing the dampened energy to be recoiled or released increasing arterial diastolic pressure which increases blood flow to vessels such as the carotids, coronary arteries, and renal arteries.

This principle has been validated in bench models showing pulse pressure reduction of 25-35% and competitive to the inventor's previous endoluminal techniques showing 30-100% left coronary blood flow improvements, and stroke volume and cardiac output improvements of up to 30%.

### IPDERD Adjustment by an Intraluminal Port Attachment Via a Femoral Artery Access Procedure 1001

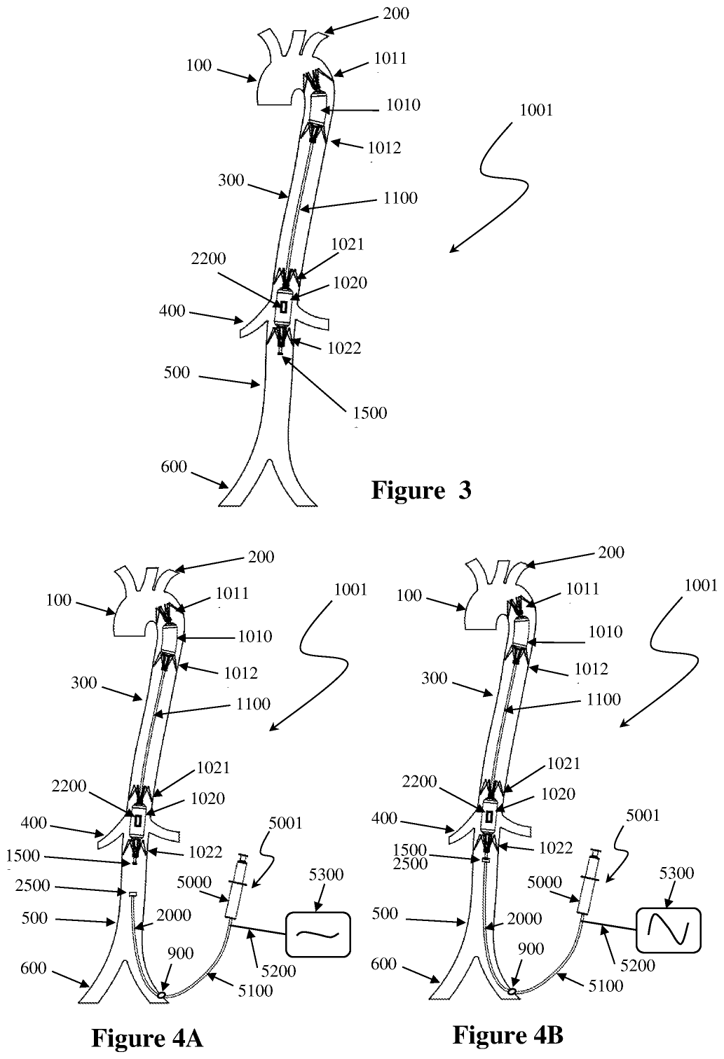

Shown in FIG. 3 is IPDERD 1001 consisting of axial balloons 1010 and 1020, connected to a catheter tubing line 1100, and fixed within the aortic vessel 100, in the upper thoracic aorta 300 distal to the left subclavian artery 200, extending to the abdominal aorta 500 adjacent to the kidneys 400, with the balloons being held by stents 1011, 1012, 1021, 1022, with the catheter tubing line 1100 having an intraluminal port 1500 attached at the distal end of the device distal to stent 1022 lower, and sensor 2200 being located within balloon 1020.

As shown in FIG. 4, inflation and adjustment of the IPDERD balloons is achieved by attaching intraluminal line 5100 with attached external pressure meter 5300 and syringe 5001, to the IPDERD intraluminal port 1500 by interventional trans-vascular access through the femoral artery 900, allowing port 2500 and line 2000 to move along aorta 500 to connect to IPDERD port 1500. The inflation and pressure adjustment can then be made. The state of the pressure in the IPDERD can also be measured. Sensor 2200) can also be used if access line 5100 has electrical connections in its lumen such as those referred to in tubing 2000.

It should be noted that FIG. 3 shows an IPDERD 1001 with an intraluminal port 1500 after the inflation and adjustment lines 2000 and 5100 have been removed and FIG. 4 shows an IPDERD being adjusted by the attachment line 2000 and 5100 being re-attached via a femoral artery access procedure using an intraluminal access line attached to the IPDERD's intraluminal port 1500 and adjusted with the attached syringe 5001 and external pressure meter 5300. During the initial deployment of the IPDERD the port 2500 and line 2500 could already by attached to facilitate deployment, and follow up adjustments could be made during planned patient reviews.

### Intraluminal Pressure Dampening and Energy Recoil Device (IPDERD) 1001

Device 1001 functions in the same way as described for device 1000 above. The performance of a fully intraluminal device, however, is limited to the amount of volume available in the balloons within the vessels. By increasing balloon diameter and length, the increased volume can increase the reduction in pulse pressure.

### IPDERD Deployment Using the Left Subclavian Artery 1002

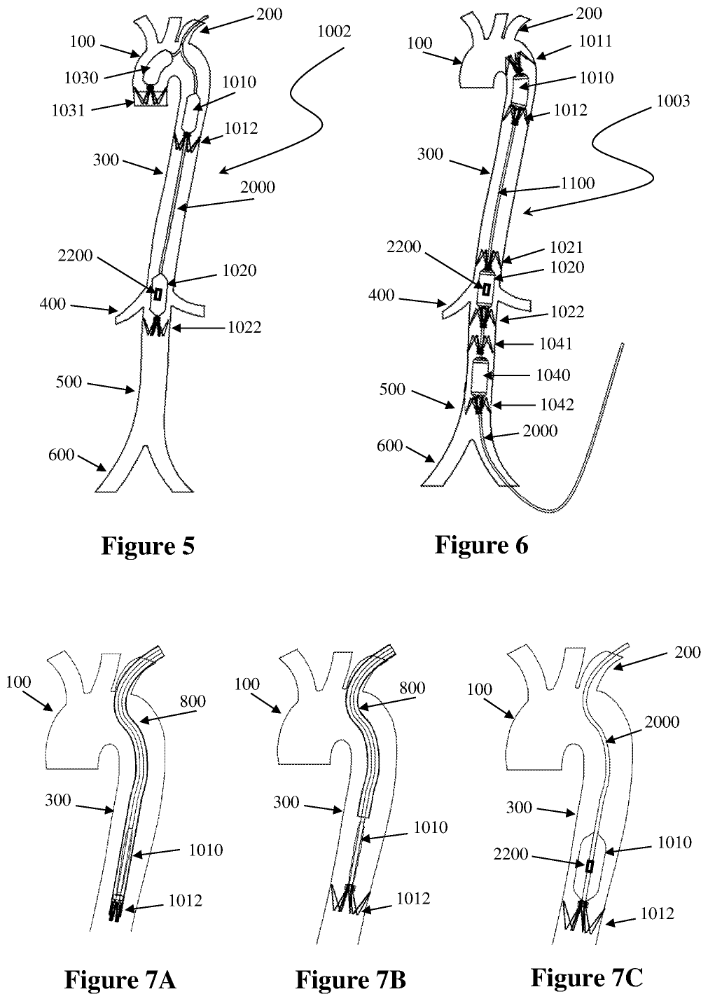

Shown in FIG. 5 is 3 balloon IPDERD deployed via the left subclavian artery with the first balloon 1030 in the ascending aorta 100, a second balloon 1010 in the thoracic aorta 300, and a third balloon 1020 in the abdominal aorta 500. A catheter tubing line is shown in the left subclavian artery (LSA) 200 which can be attached to the implantable enclosure 3000 which can be located subcutaneously adjacent the left thoracic region.

### IPDERD Deployment Using the Femoral Artery 1003

Shown in FIG. 6 is a 3 balloon IPDERD deployed via the femoral artery (FA) with the first balloon 1010 in the thoracic aorta 300 and the second 1020 and third 1030 balloons in the abdominal and lower abdominal aorta 500.

### Releasing the Crimped IPDERD in the Lumen of a Vessel

FIG. 7 shows the collapsed and crimped IPDERD being deployed in the target vessel (shown is the LSA 200) and released by the outer sheath 800 being removed allowing the stent 1012 to expand and engage the vessel wall 300, followed by the release of the balloon 1010 with and balloon inflation being made using catheter inflation line 2000.

Compressing and Loading the IPDERD into a Deployment Sheath

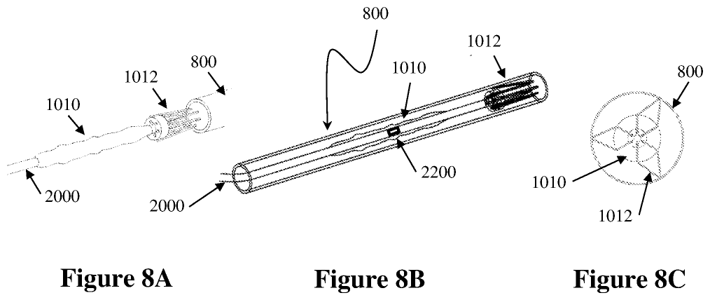

FIG. 8 shows a balloon and stent loaded into a deployment sheath.

### IPDERD Performance Comparison

A purpose-built mock circulation loop (MCL), a mechanical representation of the heart and circulatory system, was utilized for these measurements. Water at 37° C. was used as the working fluid. Pressure proximal and distal to the Intra-Aortic Balloon (IAB) device and inside the balloons were measured with calibrated gauge pressure sensors (Honeywell, Charlotte, NC, USA) and recorded with an NI data acquisition system (NI USB-6229. National Instruments. Austin, TX, USA) and labview software (Labview 2017, National Instruments, Austin, TX, USA). IAB devices with an effective balloon volume of 20 ml, 30 ml and 40 ml were placed inside a rigid simplified aorta model and fixed in position with a prototype stent structure. Prior to IAB device inflation, a gear pump (BVP-Z, Ismatec, Glattbrugg, CH) was utilized to generate a sinusoidal pulsatile flow waveform of 70 bpm as a baseline. Pump pulsation amplitude and MCL resistance were manipulated until a pulse pressure of 60 mmHg (160 mmHg systolic over 100 mmHg diastolic) was achieved. For each experiment, the IAB device was inflated until a mean pressure target of 130 mmHg inside the balloon was achieved. Pressures and flow inside the MCL were allowed to settle for 120 s prior to data collection. Pulse pressures were recorded and the pulse pressure reduction as a percentage compared to baseline was calculated. Experiments were repeated 3 times (n=3) for each balloon volume.

The IAB devices reduced pulse pressure of 60.6±1.4 mmHg on average to 49.4±0.7 mmHg, 44.6±1.9 mmHg and 38.6±0.8 mmHg for 20 ml, 30 ml and 40 ml effective balloon volume respectively. This equated to an average percentage pulse pressure reduction of 18% (20) ml balloon volume), 29% (30 ml balloon volume) and 34% (40 ml balloon volume).

When 2 IAB devices were used each having 9 ml volume to make a total of 18 ml volume, the average performance was a pulse pressure reduction of 16%, which is comparable to the single IAB of volume 20 ml.

When the 2 IAB device was then attached to an additional external balloon of 9 ml for a total volume of 27 ml, the average pulse pressure performance reduction was 24%. This demonstrates the additional performance available if extra chamber, reservoir, or balloon volume is added in the implant enclosure for a smaller length and or diameter of balloon/s used in the intraluminal components of the device.

We conclude the IPDERD prototypes are capable of reducing pulse pressures of 60.6 mmHg down to 38.6 mmHg on average using the largest tested effective balloon volume, a reduction of 34%. These results indicate the potential of the device to be a non-surgical solution for aortic recoil repair therapy for the treatment of hypertension and heart failure.

1-7. (canceled)

8. An implantable device within a tubular or sac wall of a vessel in a human or animal body, comprising:
a changeable volume portion adapted to interact with the vessel to modify the volume within the vessel;
an energy storage means functioning with the changeable volume portion, whereby a decrease in the volume of the changeable volume portion creates a pressure or energy charge in the energy storage means, the pressure or energy charge being releasable to cause the changeable volume portion to increase in volume;
axial balloons connected to a catheter tubing line;
an implantable enclosure connected to the catheter tubing line using a trans-vascular access port, the implantable enclosure comprising a subcutaneous inflation and adjustment port and an additional balloon or dampening chambers to increase the device's dampening volume;
at least one intraluminal balloon compressible and crimpable for deployment into a treatment vessel using an interventional access procedure via an adjoining vessel;
wherein the at least one intraluminal balloon is configured to be unloaded and act intraluminally within the lumen of the vessel, and the at least one intraluminal balloon and additional balloon or dampening chambers are inflatable using the subcutaneous inflation and adjustment port;
wherein when blood pressure in systole increases and travels through the vessel, a first balloon reduces its volume by compression of its media within, and higher systolic blood pressure allows this media to move downstream to connected balloons and/or chambers, reducing systolic blood pressure of the entire blood vessel system; and
wherein as arterial pressure wave propagates and diastolic phase occurs, blood pressure reduces and the first balloon expands its volume by media expanding and volume moving back from attached balloons and chambers, causing dampened energy to be recoiled or released increasing blood vessel diastolic blood pressure.

9. The implantable device of claim 8, further comprising a fixation feature for fixation of the catheter tubing line in the vessel, wherein the at least one intraluminal balloon and the fixation feature are compressible and crimpable into a deployment sheath for deployment into a vessel using an interventional access procedure via an adjoining vessel to slide the device from the access vessel to the target vessel fixation site, where the catheter tubing line, the at least one intraluminal balloon and the fixation feature are unloadable from a deployment sheath to expand the fixation feature to engage the vessel and fix the at least one intraluminal balloon and the catheter tubing line at the target vessel location.

10. The implantable device of claim 8, wherein:
the implantable device balloons and implantable enclosure balloons or chambers comprise electronic sensors;
the implantable enclosure comprises a wireless monitoring system using an electronic communication circuit to send data electronically via RF, wi-fi, or bluetooth to an external receiver;
the wireless receiver monitoring device is configured to log and record data from the implantable device; and
the implantable device sensor and wireless monitoring system are powered by an attached implanted battery, an attached implanted induction coil charged via inductive power delivered by an external coil, or via electrical power connected when an electrical subcutaneous port and power needle are used.

11. The implantable device of claim 8, wherein the implantable device operational pressure or charge is adjustable to the mean pressure of the vessel using the enclosure subcutaneous inflation and adjustment port and a subcutaneous needle and syringe.

12. The implantable device of claim 9, wherein:
the inflation and adjustment line is removable after implantation;
the device catheter tubing line uses a connected intraluminal port;
inflation and adjustment of the device dampening volume is achievable by re-attaching an intraluminal line with an intraluminal line port, using an interventional trans-vascular access through an adjoining vessel;
the intraluminal line port and intraluminal line are movable along the vessel to connect to the device's intraluminal port for adjusting the device mean pressure; and
the intraluminal line port and intraluminal line are removable after adjustment.

13. The implantable device of claim 8, wherein the at least one intraluminal balloon is implantable via femoral artery access or subclavian access, into one or more of: an ascending aorta, a thoracic aorta, an abdominal aorta, or a lower abdominal aorta.

14. The implantable device of claim 8, wherein the implantable device reduces pulse pressure by 16% to 34% by using the implantable device dampening volume of 18 milliliters to 40 milliliters.

15. A method of treating a vessel in a human or animal body, comprising:
preparing a patient;
identifying a site in the vessel requiring treatment;
positioning an implantable device within a portion of tubular or sac wall of the vessel at the site, whereby load applied to the vessel is borne by the wall and the device, the vessel being assisted by the device when the wall and the device acts upon the load, the device comprising:
an energy storage means which is charged with a pressure or energy charge by means of the load being applied to the device;
axial balloons connected to a catheter tubing line;
an implantable enclosure connected to the catheter tubing line using a trans-vascular access port, the implantable enclosure comprising a subcutaneous inflation and adjustment port and an additional balloon or dampening chambers;
one, two, or three intraluminal balloons;
deploying the intraluminal balloons in a crimped or collapsed state to the vessel treatment site where they are unloaded and act intraluminally within the lumen of the vessel;
inflating the intraluminal balloons and additional balloon or dampening chambers using the subcutaneous inflation and adjustment port;
wherein when blood pressure in systole increases and travels through the vessel, a first balloon reduces its volume by compression of its media within, and higher systolic blood pressure allows this media to move downstream to connected balloons and/or chambers, reducing systolic blood pressure of the entire blood vessel system; and
wherein as arterial pressure wave propagates and diastolic phase occurs, blood pressure reduces and the first balloon expands its volume by media expanding and volume moving back from attached balloons and chambers, causing dampened energy to be recoiled or released increasing blood vessel diastolic blood pressure.

16. The method of claim 15, further comprising:
compressing and crimping a fixation feature and the intraluminal balloons into a deployment sheath;
deploying the device into a vessel using an interventional access procedure via an adjoining vessel to slide the device from the access vessel to the target vessel fixation site;
unloading the catheter tubing line, the intraluminal balloons and the fixation feature from the deployment sheath to expand the fixation feature to engage the vessel and fix the intraluminal balloons and catheter tubing line at the target vessel location.

17. The method of claim 15, further comprising:
monitoring the implantable device using electronic sensors in the implantable device balloons and implantable enclosure balloons or chambers;
sending data electronically from a wireless monitoring system in the implantable enclosure to an external receiver;
logging and recording data from the implantable device using a wireless receiver monitoring device; and
adjusting the implantable device operational pressure or charge to the mean pressure of the vessel using the enclosure subcutaneous inflation and adjustment port and a subcutaneous needle and syringe, using the sensor and wireless monitoring system or an external pressure monitor.

18. The method of claim 17, further comprising powering the implantable device sensor and wireless monitoring system by one of: an attached implanted battery; an attached implanted induction coil charged via inductive power delivered by an external coil; or electrical power connected when an electrical subcutaneous port and power needle are used.

19. The method of claim 16, further comprising:
removing the enclosure and inflation port after implantation;
connecting an intraluminal port to the device catheter tubing line;
achieving inflation and adjustment of the device dampening volume by attaching an intraluminal line with an intraluminal line port, using interventional trans-vascular access through an adjoining vessel;
moving the intraluminal line port and intraluminal line along the vessel to connect to the intraluminal port of the device;
adjusting the device mean pressure with an attached syringe and external pressure meter or using a balloon sensor; and
removing the intraluminal line port and intraluminal line after adjustment.

20. The method of claim 15, wherein implanting the device comprises using interventional techniques via femoral artery access or subclavian access to implant the device into one or more of: the ascending aorta, thoracic aorta, abdominal aorta, or lower abdominal aorta.

21. The method of claim 15, wherein the implantable device reduces pulse pressure by 16% to 34% by using the implantable device dampening volume of 18 millilitres to 40 millilitres.
